(19) United States
(12) Patent Application Publication
(10) Pub. No.: US 2021/0196749 A1
(43) Pub. Date: Jul. 1, 2021

(54) AQUEOUS HYPOHALOUS ACID PREPARATIONS FOR THE INACTIVATION OF RESISTANT INFECTIOUS AGENTS

(71) Applicant: BRIOTECH, INC., Woodinville, WA (US)

(72) Inventors: Daniel James TERRY, Woodinville, WA (US); Jeffrey Francis WILLIAMS, Woodinville, WA (US)

(73) Assignee: Briotech, Inc., Woodinville, WA (US)

(21) Appl. No.: 17/047,054

(22) PCT Filed: Jun. 12, 2019
(86) PCT No.: PCT/US2019/036722

§ 371 Date: Oct. 12, 2020

Related U.S. Application Data
(60) Provisional application No. 62/656,513, filed on Apr. 12, 2018.

(51) Int. Cl. A61K 33/20; A61P 31/22

(52) U.S. Cl. CPC A61K 33/20; A61P 31/22

(57) ABSTRACT

Methods for the inactivation of highly resistant infectious agents, on inanimate or epithelial surfaces or in suspension, upon exposure to aqueous solutions or gels containing hypohalous acids, and the use of these preparations in the treatment, prevention, and interruption of transmission of contagious diseases, particularly infections of oral and genital mucosal and mucocutaneous epithelial surfaces.

### CROSS-REFERENCE TO RELATED APPLICATION

This application claims the benefit of U.S. Application No. 62/656,513, filed Apr. 12, 2018, expressly incorporated herein by reference in its entirety.

### BACKGROUND

### Technical Field

The present disclosure relates generally to methods for the inactivation of highly resistant infectious agents, on inanimate or epithelial surfaces or in suspension, upon exposure to aqueous solutions or gels containing hypohalous acids, and the use of these preparations in the treatment, prevention, and interruption of transmission of contagious diseases, particularly infections of oral and genital mucosal and mucocutaneous epithelial surfaces.

### Description of the Related Art

The resurgence of infectious diseases worldwide has been accompanied by the selection of many infectious microbes that display multi-drug resistance traits, and by the recognition that the commonly applied chemical disinfection and sterilization methods can no longer be relied upon to inactivate all invasive agents. Ineffective decontamination has been shown to allow for transmission of resistant viruses and self-replicating proteins (prions) by medical instruments. Recently improved means of detection of these agents in bodily fluids and on medical and dental devices have raised the specter of inadvertent exposure of patients and healthcare workers to diseases caused by, for example, human papilloma viruses (HPV, causal in cervical and oropharyngeal cancer), and neurodegenerative conditions such as Creutzfeldt Jakob Disease (CJD) and Alzheimer's Disease (AD).

Conventional compositions and methods for high level disinfection of inanimate surfaces or contaminated epithelia are not sufficient for the inactivation of all these infectious agents. They require long and impractical exposure times, or hazardous or corrosive solutions or vapors that cannot be used on expensive instruments or on living tissues. They therefore do not provide ready solutions to growing health risks from drug and disinfectant resistant agents.

In general, formulations containing hypochlorous acid (HOCl) along with other aqueous forms of chlorine are effective antimicrobial agents with proven antiviral, antibacterial, antifungal, and antiprotozoal properties that are useful in disinfection measures applied in human and animal health, and horticulture. Although HOCl is unstable and impure when produced under conventional conditions, crude mixtures containing HOCl may be generated on-site for short-term applications in all these fields of use. More exacting manufacturing processes can furnish pure HOCl with stability that permits prolonged storage, even for periods up to several years enhancing its utility for many professional, consumer and industrial applications. HOCl is the conjugate acid of hypochlorite (OC1′), and is produced naturally in pure form in vivo by neutrophils in mammals, and in the circulating monocytes and tissue-resident macrophages and microglial cells to inactivate pathogens within phagocytic vesicles and in the extracellular space around phagocytes in tissues. HOCl solutions are weakly acidic, in contrast to the high alkalinity of household hypochlorite bleach (˜pH 12); preparations of HOCl are therefore compatible with applications for which bleach is damaging and hazardous to users, and to surfaces to which it is applied. Pure HOCl formulations in the form of BrioHOCl™ (Briotech, Inc., Woodinville, Wash.) can be applied directly to the skin and mucous membranes, and used as cosmetics, and as topical therapeutics for humans and domestic animals. HOBr is the conjugate acid of hypobromite, and is produced naturally in eosinophils of mammals via enzymatic pathways similar to those used to generate HOCl, except that intracellular bromide ion is oxidized to HOBr rather than chloride ion in the case of HOCl. HOBr has a pKa higher than HOCl. This permits its availability in solution at pH levels higher than those suitable for HOCl.

HOCl molecules in water are neutral, but aqueous solutions maintain a high positive Oxidation-Reduction Potential (ORP), demonstrable by insertion of millivoltmeter electrodes that will register my potentials in the 1100+ range for BrioHOCl™, for example. The measurement of ORP has become accepted as an indicator of the disinfecting capability of active chlorine solutions. The extreme reactivity of HOCl leads to known and rapid interactions with a wide range of chemical groups, including oxidation and chlorination reactions with amino acids, lipids and sulfur-containing structures. Many different possibilities arise as to the mechanisms of antimicrobial activity expressed by HOCl solutions, but specific means whereby the infectivity of any particular pathogen is destroyed remain unknown. Nonetheless, there is ample evidence of sites of vulnerability to HOCl in a wide range of proteins described in the primary biochemistry literature. This makes it reasonable that HOCl should interact with those specific sites when they are expressed in proteins that make up important constituents of infectious agents of concern in contemporary healthcare, such as in the capsids of resistant small non-enveloped viruses (e.g., HPV) or infectious proteins themselves.

HOCl and HOBr are known to express a potency in chemical and anti-infective agent reactions that rises to two or more orders of magnitude higher than that of the corresponding hypochlorite and hypobromite entities found in aqueous solutions at pH levels in the extreme alkaline range. Hypochlorite and hypobromite solutions are used for decontamination against a wide range of pathogens, including bacterial spores, non-enveloped virus particles (some of which are amongst the most difficult to inactivate agents), protozoan oocysts, and even prions that function as infectious proteins. Thus, prolonged incubation of prion contaminated items in concentrated sodium hypochlorite bleach is accepted as a disinfecting measure for this purpose. Likewise, hypobromite solutions have been shown to have inactivation efficacy against prion proteins responsible for bovine transmissible spongiform encephalopathy (TSE, also known as Mad Cow Disease). However, extended exposure of inanimate objects to corrosive solutions of hypochlorite or hypobromite causes damage that may make the practice entirely unacceptable, or cause it to be applied only reluctantly. Similarly, the corrosive effects of these solutions are hazardous to users, and contribute to the unwillingness to use these effectors of inactivation routinely in healthcare institutions and other settings.

At the other end of the scale, acidified electrolyzed solutions of sodium chloride contain aqueous chlorine species that have been shown to have rapid and high level inactivation capacities for a wide range of infectious particles, including bacterial and fungal spores; there is demonstrable activity against infectious prion proteins of Creutzfeld Jacob Disease (CJD). However, at the extremes of pH (2.6) used for these prion decontamination procedures, there is a predominance of aqueous elemental chlorine as the major oxidant, along with hydrochloric acid (HCl) and some hypochlorous acid. It has been determined that most of the oxidant efficacy under these conditions is attributable to elemental chlorine. The anti-prion efficacy of these formulations is therefore also by inference a function of aqueous chlorine itself. There are hazards associated with the production and handling of this product, including to personnel, on top of the associated presence in the formulation of hydrochloric acid.

The efficacy of extreme alkaline or acidic solutions versus prions has attracted interest because of their emerging significance as causes of an increasing number of neurodegenerative disorders in animals and man. Prion diseases, or transmissible spongiform encephalopathies (TSEs), are fatal, untreatable, and transmissible neurodegenerative diseases of many mammalian species. In humans, prion diseases include sporadic, variant and genetic forms of Creutzfeldt-Jakob disease (sCJD, vCJD and gCJD) as well as a number of other disorders. Prion diseases of other species include classical bovine spongiform encephalopathy (cBSE), scrapie in sheep, goats and rodents, and chronic wasting disease of cervids. All mammalian prion diseases share an underlying molecular pathology that involves the conversion of the hosts' normal form of prion protein, (e.g., PrP^{C}), to a misfolded, aggregated, infectious and pathological form (e.g., PrP^{Sc}).

There is recent recognition that pathological forms of proteins that become altered in their conformation are associated with a wider spectrum of diseases than those classically recognized as resulting from transmissible prions, such as CJD, BSE, Scrapie and CWD. Thus, now included in the list of diseases that may result from conformationally-altered or misfolded proteins are Alzheimer's Disease, Parkinson's Disease, Frontotemporal Dementia and other neurodegenerative disorders, along with Diabetes Type II, Multiple Systemic Atrophy, and other conditions in which identifiable, abnormally folded proteins may be causative.

All these prions are unusual, compared to other types of pathogens, in that they lack a pathogen-specific nucleic acid genome, and are particularly resistant to biochemical, chemical, physical (e.g., heat, U/V light) or radiological inactivation. As a result, prions resist complete inactivation under conditions that are typically used in healthcare, the food industry, and agriculture to inactivate other types of disease agents. Indeed, current recommendations are that extremely harsh chemical treatments such as 1-2 N sodium hydroxide, 20-40% household bleach (˜12,000-24,000 mg/L sodium hypochlorite), prolonged (up to 60 min) autoclaving at an unconventionally high temperature of 132° C. and/or prolonged exposure to incinerator temperatures be used to decontaminate biological materials or solid surfaces that may be contaminated with prions. An anti-prion reagent that was developed and registered with the USEPA as a commercial disinfectant (Environ™ LpH™, an acidic phenolic disinfectant) proved impractical for wide use, and was removed from the U.S. market.

In general, it has been determined that all such treatments may not only be hazardous to the user, but can also be incompatible with, or not applicable to, instruments, equipment or surfaces that may require prion decontamination. Similar constraints have emerged in regard to disinfection of surfaces bearing resistant non-enveloped viruses, such as Human Papilloma Viruses (HPV). In that instance there are currently widespread concerns regarding the use of instruments that may provide an infection source for oral or genital agents that can lead to malignant cancerous lesions. There is therefore an urgent need for effective high level chemical decontamination methods that are more safely and broadly applicable to the entire spectrum of resistant infectious agents, including resistant viruses and transmissible proteins. The availability of effective, practical inactivation methods for routine use on potentially contaminated tools, instruments and environmental surfaces would seriously reduce the risks of iatrogenic disease transmission, including during the commonly practiced procedures for monitoring HPV associated lesions in the genital tract of women.

Concentrated corrosive solutions, such as lye, or concentrated household bleach act only slowly to degrade the infectivity of resistant agents that take the form of proteins. Certain less corrosive solutions, such as aldehydes, phthalaldehydes, alcohols, and phenols that are considered by U.S. and international regulatory agencies as chemical sterilants and are approved for use as such, are now known not to be effective against certain resistant organisms, including HPV.

Despite the advances made in the inactivation of disease agents, a need exists for convenient, cost-effective, entirely non-hazardous methods applicable to high level decontamination/inactivation of disease agents that pose challenges for infection control measures today. The present disclosure seeks to fulfill this need and provides further related advantages.

### BRIEF SUMMARY

The present disclosure provides for convenient, cost-effective, entirely non-hazardous methods applicable to high level decontamination/inactivation of resistant forms of agents that pose challenges for infectious disease control measures today. The methods do not result in damage to surfaces, devices, equipment, and do not require heat or prolonged exposures to or immersion in toxic or corrosive solutions or vapors. The preferred aqueous formulations disclosed herein are sufficiently safe and non-toxic to allow for application at full strength to delicate and expensive medical/dental instruments, contaminated environmental surfaces, and to human skin and mucosal surfaces that are subject to localized infections, and are not readily either treated or prevented by currently available options.

The disclosure provides a method for inactivating an infectious agent that is a resistant virus, a cancer-causing virus, a chemically-resistant non-enveloped virus, or an infectious agent, present in a mucous membrane or epithelial surface. In the method, the infectious agent is contacted with a bufferless, electrolyzed, hypohalous acid composition.

In certain embodiments, the infectious agent is an infectious microbe, such as a virus, a bacterium, a fungus, or a protozoa.

In other embodiments, the infectious agent is an infectious protein, such as a self-replicating protein or a prion. In certain of these embodiments, the prion is an agent of Creutzfeldt Jakob Disease, Bovine Spongiform Encephalopathy, Chronic Wasting Disease, Scrapie, Alzheimer's Disease, Parkinson's Disease, or Amyotrophic Lateral Sclerosis.

In certain embodiments, the infectious agent is a microbial pathogen, such as a Gram negative bacterium or a Gram positive bacterium. In certain of these embodiments, the microbial pathogen is Acinetobacter baumannii, Escherichia coli, Escherichia coli 0157 Pseudomonas aeruginosa, Salmonella choleraesuis, Shigella flexneri, Escherichia coli NDM-1, Klebsiella pneumonia, Yersinia enterocolitica, Proteus vulgaris, Neisseria, Chlamydia, or Listeria, or the microbial pathogen is Bacillus subtilis, Staph epidermidis, MRSA (Staph. aureus), Enterobacter cloacae, or Enterococcus VRE.

In certain embodiments, the microbial pathogen is a fungus, such as Candida albicans or Aspergillus niger.

In other embodiments, the microbial pathogen is a virus, such as Coronavirus [Human, OC43], Human Papilloma Virus (HPV), or MS-2.

In certain embodiments of the above methods, the composition has been thermally stressed prior to use, or has been stored for a prolonged period of time prior to use.

In certain embodiments of the above methods, the composition further comprises stabilizers.

The composition useful in the above methods can be formulated as a solution, or a spray, a fog, a mist, or an aerosol of droplets (e.g., micronized droplets in the submicron size range and aerosolized droplets), or a gel, or a viscous liquid.

In certain embodiments of the above methods, contacting with the composition comprises contacting from one second to several hours.

In certain embodiments of the above methods, contacting with the composition comprises contacting at room temperature.

In certain embodiments of the above methods, contacting with the composition comprises contacting at a temperature in the range from about room temperature to about 80° C.

In certain embodiments of the above methods, the hypohalous acid composition is a hypochlorous acid composition. In certain of these embodiments, the hypohalous acid composition is an aqueous hypochlorous acid composition having a hypochlorous acid concentration from about 5 to about 500 mg/L, a pH from about 3.2 to about 6.0, an oxidative reduction potential (ORP) of about +1000 millivolts, and containing from about 0.85% to about 2.0% by weight chloride salt based on the total weight of the composition. In certain of these embodiments, the hypohalous acid composition is an aqueous hypochlorous acid composition having a hypochlorous acid concentration from about 80 to about 300 mg/L, a pH from about 3.8 to about 5.0, an oxidative reduction potential (ORP) of about +1100 millivolts, and containing from about 0.85% to about 2.0% by weight chloride salt based on the total weight of the composition. In certain of these embodiments, the hypohalous acid composition is an aqueous hypochlorous acid composition having a hypochlorous acid concentration from about 80 to about 300 mg/L, a pH from about 4.0 to about 4.3, an oxidative reduction potential (ORP) of about +1138 millivolts, and containing from about 0.85% to about 2.0% by weight chloride salt based on the total weight of the composition.

In other embodiments of the above methods, the hypohalous acid composition is a hypobromous acid composition. In certain of these embodiments, the hypohalous acid composition is an aqueous hypobromous acid composition having a hypobromous acid concentration from about 10 to about 300 mg/L, a pH from about 3 to about 8.5, an oxidative reduction potential (ORP) of about +1000 millivolts, and containing from about 0.85% to about 2.0% by weight chloride salt based on the total weight of the composition. In certain of these embodiments, the hypohalous acid composition is an aqueous hypobromous acid composition having a hypobromous acid concentration from about 5 to about 350 mg/L, a pH of about 7 to about 8, an oxidative reduction potential (ORP) of about +900 millivolts, and containing from about 0.85% to about 2.0% by weight chloride salt based on the total weight of the composition.

The composition useful in the above methods can include a chloride salt, such as an aqueous soluble chloride salt selected from sodium chloride, potassium chloride, magnesium chloride, and ammonium chloride. In certain embodiments, the chloride salt is sodium chloride. In certain embodiments, the composition contains about 2.0% by weight chloride salt based on the total weight of the composition. In certain of these embodiments, the composition contains about 2.0% by weight sodium chloride based on the total weight of the composition.

In certain embodiments of the above methods, the composition does not contain a detectable amount of aqueous oxidative chlorine other than a hypohalous acid.

### BRIEF DESCRIPTION OF THE SEVERAL VIEWS OF THE DRAWINGS

The foregoing aspects and many of the attendant advantages of this disclosure will become more readily appreciated as the same become better understood by reference to the following detailed description, when taken in conjunction with the accompanying drawings.

FIG. 1 is a graph illustrating data on HPV disinfection with conventional agents.

FIG. 2 is a graph illustrating data on HPV disinfection with BriotechHOCl™.

### DETAILED DESCRIPTION

Methods are disclosed for the practical, safe, convenient, high-level inactivation of disinfection-resistant infectious agents using hypohalous acid formulations. The optimal method is preferably implemented by the exposure of infectious agents for periods ranging from 15 seconds up to 60 minutes at 20-50° C., to aqueous solutions or gels of electrolytically-generated hypochlorous acid (HOCl), or to solutions or gels of electrolytically—then chemically-generated hypobromous acid (HOBr), or chemically-generated forms of these hypohalous acids. Such preparations rapidly and potently inactivate infectious agents in the most chemically and physically resistant categories known to microbiological science. They bring about irreversible changes in the structure, conformation and infectivity of a wide range of resistant viruses, bacteria, fungi, and even infectious proteins (prions). Pure preparations of these hypohalous acids can be made that are innocuous to human skin and mucous epithelia, including in the oral, respiratory and genital tracts. The power of these compositions and their benign profiles as safe, innocuous preparations compatible with skin and mucosal epithelial surfaces provide novel means of decontamination of both inanimate surfaces and epithelial membranes. Their use opens up novel interventions aimed not only at interruption of transmission of many resistant infectious agents of humans and animals, but also new means of treating localized infections that they cause, limiting their release into mucosal fluids, and enabling prevention of exposure of susceptible hosts. Their use as chemical disinfectants and sterilants offers new methods of achieving safe, rapid decontamination of environmental surfaces, instruments, and exposed personnel, and their protective clothing and devices so as to improve infection control measures in human and animal healthcare.

Methods are disclosed for inactivating infectious agents to a high degree, after short exposure periods, and under conditions that are mild and harmless to surfaces, instruments, equipment, epithelial surfaces of infected patients or healthcare personnel, such as skin and oral, respiratory and genital mucosae. These methods are strikingly different in character and duration from those conventionally applied to the decontamination of items and surfaces that are suspected of containing or having been exposed to highly resistant agents such as resistant viruses or infectious proteins (e.g., prions).

In the past, suitable levels of confidence in the complete inactivation of all infectious agents required harsh and prolonged high temperature treatments (for example, using pressurized steam at 132° C. for 30 minutes) after prior immersion in caustic and corrosive chemical agents such as 2N sodium hydroxide or concentrated sodium hypochlorite solutions (10,000-40,000 mg/L), or peracetic acid for periods of 1-2 hours. These procedures create significant hazards to personnel handling large volumes of dangerous chemical solutions, and exposing costly autoclaving equipment to vapors created by extensive heat treatment of the decontamination target.

By contrast, the methods disclosed herein allow inactivation of resistant agents at room temperature (20° C.), in short contact periods (seconds to an hour) without necessity for additional, high temperature post-chemical exposure treatment. The methods disclosed herein do not involve expensive, corrosive or impractical compositions or procedures. Prior methods, while proven to degrade the infectivity of all known agents do not readily find a place in the real-world practice of high level decontamination in healthcare or other arenas, such as carcass preparation and food processing.

The inactivating constituents in the method disclosed herein are preferentially aqueous solutions of a hypohalous acid (hypochlorous acid, or hypobromous acid) in which the contaminated article or tissue or bodily fluid is suspended for periods up to one hour at 20° C. in order to achieve reductions in infectivity of 6 LRV or greater. Exposure to these preparations at higher temperatures can beneficially accelerate the inactivation process and are shown not to be deleterious to the inactivating principles in the formulations. The hypohalous acid concentrations required for maximal inactivation are optimally in the 150-300 mgs/L range. Lesser concentrated solutions, or exposures for shorter periods, can nonetheless result in significant reductions in the infectivity of target agents. At these optimal concentrations the inactivating formulations are not corrosive or toxic to mammalian cells in vitro, or to human or animal skin or mucous membranes, including nasal, oral and genital epithelia. These specifications for effective degradation of the infectious potential of highly resistant microbial agents, such as bacterial and fungal spores, and non-enveloped, capsid-protein coated viruses and even infectious proteins, are compatible with practical demands of healthcare and environmental disinfection and decontamination. They permit adoption of the disclosed methods for widespread use in combatting transmission of all resistant disease agents. They are compatible with commercial viability of the methods for everyday use, without concerns for the integrity and utility of treated surfaces, devices, and equipment, or for the health and safety of personnel responsible for executing the methods on a routine basis.

The methods disclosed herein also provide the advantage that high level decontamination can be accomplished in one step for resistant spores and viruses and multi-drug resistant vegetative forms of microbial disease agents and infectious proteins. This is in contrast to previous chemical approaches that required addition of conventional disinfecting or denaturing formulations or procedures after the primary exposure to decontamination measures.

The methods disclosed herein also permit methods of exposure of contaminated surfaces, equipment, devices, clothing or personnel to inactivating formulations that are based upon the misting or vaporization of the hypohalous acid into confined spaces. This procedure ensures dispersion of the active agents into crevices and microenvironments, even onto personnel who are suspected of having been contaminated by infectious tissues or bodily fluids. The exposures can be affected without concerns for toxicity or corrosiveness which accompany prior methods of inactivation of highly contagious and resistant infectious agent types. Vaporization of these formulations may enable beneficial therapeutic or prophylactic impacts on resistant viral, bacterial or fungal infections.

While the preferred methods of inactivation disclosed herein make use of aqueous solutions of hypohalous acids at concentrations in the 150-300 mg/L range, the active constituents are compatible with formulations as gels or viscous fluids which may be applied to target surfaces, either inanimate or representative of the infected epithelial mucosal or skin surfaces of infected persons or animals, so as to ensure prolonged and intimate contact with the necessary levels of active halogen species. The active species may also be dispersed into the air in confined spaces as a mist in order to achieve environmental disinfection at a high level.

The overall aspect of the preferred methods of inactivation disclosed herein is the exposure of targeted surfaces, equipment, devices, tissues or bodily fluids to solutions of hypochlorous acid within the range of pH 3.9-5.9, with an Oxidation Reduction Potential (ORP) of +1140 millivolts (my) containing no less than 0.9% NaCl for periods up to one hour. Variations of these specifications may allow for preferential use on sensitive mucosal surfaces, for example, that optimizes acceptability to the exposed patient or animal.

A further advantage of the disclosure is the suitability of the inactivation methods for treatment of potentially contaminated tissues that may be useful in transplantation procedures such as corneal grafting, dura grafts, or other tissues or organs that may be suitable for restoration of functions in a recipient host, or may be used for cosmetic manipulation of the recipient (e.g., bovine collagen injections or implants).

A further advantage of the disclosure is the suitability of the inactivation methods for the pre-treatment of implanted devices, electrodes, sensors, and the like into the human body for the purposes of restoring or assisting in preservation of functions in the recipient host.

A further advantage of the disclosure is the suitability of alternative means of generating the hypohalous acid active principles, other than the preferred means used herein for the electrolytic generation of hypohalous acids. Hypohalous acids can be formed by judicious adjustments of pH and other modifications of solutions containing precursor aqueous halogen species, either free or combined in the form of organic heterocyclic compounds with affinities for active oxidizing halogens. These optional approaches and others will be apparent to those skilled in the art upon reading details of the efficacy of hypohalous acid decontaminating formulations in the examples described below.

### Hypohalous Acid Compositions

The methods of the disclosure utilize a hypohalous composition that is a bufferless, electrolyzed, hypohalous acid compositions.

In certain embodiments, the bufferless, electrolyzed, hypohalous acid composition, comprises a hypohalous acid and a chloride salt in an amount from about 0-to about 2.0% by weight based on the total weight of the composition. In certain of these embodiments, the chloride salt is an amount from about 0.85 to about 2.0% by weight based on the total weight of the composition.

In certain embodiments, the hypohalous acid is hypochlorous acid.

In certain of these embodiments, the composition comprises hypochlorous acid at a concentration from about 5 to about 500 mg/L, and has a pH from about 3.2 to about 6.0, and an oxidative reduction potential (ORP) of about +1000 millivolts.

In other of these embodiments, the composition comprises hypochlorous acid at a concentration from about 80 to about 300 mg/L, and has a pH from about 3.8 to about 5.0, and an oxidative reduction potential (ORP) of about +1100 millivolts.

In further of these embodiments, the composition comprises hypochlorous acid at a concentration from about 80 to about 300 mg/L, and has a pH from about 4.0 to about 4.3, and an oxidative reduction potential (ORP) of about +1138 millivolts.

In other embodiments, the hypohalous acid is hypobromous acid.

In certain of these embodiments, the composition comprises hypobromous acid at a concentration from about 10 to about 300 mg/L, and has a pH from about 3 to about 8, and an oxidative reduction potential (ORP) of about +1000 millivolts.

In other of these embodiments, the composition comprises hypobromous acid at a concentration from about 5 to about 350 mg/L, and has a pH of about 7, and an oxidative reduction potential (ORP) of about +900 millivolts.

In certain embodiments, the chloride salt is an aqueous soluble chloride salt selected from sodium chloride, potassium chloride, magnesium chloride, and ammonium chloride. In certain embodiments, the chloride salt is sodium chloride.

The HOCl compositions do not contain a detectable amount of aqueous oxidative chlorine other than HOCl. The HOBr compositions do not contain a detectable amount of aqueous oxidative bromine other than HOBr.

In certain embodiments, the hypohalous acid is hypochlorous acid and the composition has a shelf life of useful inactivation efficiency up to about 5 years in a sealed container.

In other embodiments, the hypohalous acid is hypobromous acid and the composition has a shelf life of useful inactivation efficiency of from about four to about six weeks in a sealed container.

The hypohalous acid composition does not include a hypohalous acid stabilizer. The hypohalous acid composition does not include a mono- or di-phosphate sodium or potassium buffer, a carbonate buffer, periodate, divalent metal cation, organic heterocyclic compound, hydrochloric acid, hydrobromic acid, or a chemical entity conventionally used as a halogen stabilizer to enhance the stability of a hypohalous acid solution in storage.

The composition may be formulated to suit the desired application. In certain embodiments, the composition is formulated as a solution, a spray or fog or mist or aerosol of droplets (e.g., micronized droplets in the submicron size range and aerosolized droplets), a gel, or a viscous liquid.

The embodiments noted above are bufferless aqueous compositions. It will be appreciated that in other embodiments of the above-described compositions, the aqueous compositions include one or more buffers.

Representative hypohalous acid compositions useful in the practice of the present disclosure are described in WO 2107/223361 (PCT/US2017/038838), expressly incorporated herein by reference in its entirety.

The following examples are provided for the purpose of illustrating, not limiting, the disclosure.

### EXAMPLES

The following examples are set forth to provide those of skill in the art with a new methods of use of hypohalous acids for the inactivation resistant microbial agents, and the means of detection and measurement of the effectiveness of these methods in bringing about such inactivation.

### Materials and Methods

BrioHOCL™ was supplied by Briotech Inc., Woodinville, Wash. Briefly, HOCl results from electrolysis of an aqueous solution of sodium chloride so as to provide at the anode conditions that attract and stabilize reaction products that form HOCl. The end-product is a solution with a range of pH on packaging and storage of 4-6 at warehouse environmental temperatures (3.5° C. to 35° C.), an ORP of +1100 my, a salt (NaCl) concentration of either 0.9% or 1.8% by weight, and a free chlorine concentration of 250-300 mg/L at the time of production.

Hypobromous acid (HOBr) was prepared by the exposure of one equivalent of aqueous bromide ion (as NaBr) to one equivalent of unbuffered electrolytically-generated HOCl. This solution was prepared fresh for use in tests for inactivation of highly resistant microbial organisms.

In some experiments modifications to these solutions were made by the addition of buffering stabilizers or other organic heterocyclic compounds known to enhance the stability of oxidative chlorine in aqueous solution.

Physical and chemical characterization of anti-infectious agent formulations based on hypohalous acids is described below.

### Active Chlorine Measurement

Hach reagent kits for Total Chlorine (Hach Company, Loveland, Colo.) were used for determination of the active Cl content of the BrioHOCl™ formulation, after validation by comparison of manual iodometric and digital titration results on 33 samples (six replicates each). Thereafter the digital Hach device was used (4 replicates per sample) to measure active Cl in all samples used for antimicrobial efficacy testing.

The pH, Oxidation Reduction Potential (ORP in my) and conductivity were recorded for all samples using a Hach Multi Parameter meter (Model HQ40d). ORP targeted at production was +1140 my, at pH 3.9. Starting active Cl concentrations were varied in production lots during electrolysis, depending on intended applications. Generally, these values ranged between 175 and 350 ppm active Cl, with background NaCl concentrations of either 0.9 or 1.8%.

### UV/Vis Spectrophotometry

Test solutions were loaded into 1 mL quartz cuvettes, and spectra obtained using a BioMate 3S UV-Visible Spectrophotometer. The instrument was blanked using Nanopure water, and test solutions consisted of undiluted BrioHOCl™ at selected time points in the sequential sampling of product stored at room temperature. Absorbance was measured from 190 to 400 nm, with peak absorbance for HOCl registered at 238 nm. Test solutions of HOBr showed an absorbance peak at 260 nm, with no detectable presence of HOCl 5 minutes after the addition of NaBr.

### Raman Spectroscopy

Spectra were obtained using a Renishaw InVia Raman microscope. Spectra were observed using an excitation wavelength of 785 nm with undiluted BrioHOCl™ in a 1 mL quartz cuvette. The acquisition time for each scan was 20 seconds, and 100 acquisitions were accumulated. A deionized water blank was scanned in the same manner, and the background produced was subtracted from the test sample data using Igor software.

### HPV Disinfection Protocol

These studies were performed using HPV16, the most important and common cause of cervical and oral cancer. Virus particles (50 11.1, 2-5×10⁶ virus) were generated and tested. HOCl solutions were provided by Briotech Inc. Efficacy was measured in a suspension assay with HPV16 incubated with each concentration of the disinfectant at ambient temperature (room temperature) for 5, 10, 20, 30, and 45 minutes. The neutralizer solution consisted of 0.1% Tween 80; 1% peptone; 1% cysteine; 0.5M Tris buffer (pH 7.5). Controls: (A) virus with no disinfectant (50 11.1, 2-5×10⁶ virus) plus media (no disinfectant), (B) hypochlorite. All assays were done in triplicate.

Following contact time and addition of neutralizer the solutions were centrifuged in an Amicon Ultra centrifugal filter 100,000 MW cut-off (MWCO) (Millipore) at 4,000 rpm for 10 min, and then treated with more neutralizer and recentrifuged at 4,000 rpm for 10 min. After two more washes with HaCaT culture media or until pH indicator shows physiological pH levels, the resulting liquid contains the treated virus and was collected and assayed for infectivity. For this purpose HaCaT cells are seeded (5×10⁴ cells/well) into 24-well cell culture plates in 0.5 ml HaCaT media [1] and grown to confluence (48 hours) at 37° C./5% CO₂.

After 48 hours, the media were aspirated and disinfectant-treated virus added to HaCaT cells. Virus and cells were incubated for 48 h at 37° C./5% CO₂.

The ability to infect the HaCaT cells after 48 h of incubation was determined by the presence of the spliced HPV16 E1\hat{ }E4 mRNA species. Total mRNA is harvested with the SurePrep True Total RNA Purification Kit (Fisher Scientific) and diluted to a final concentration of 100 ng/μ1 with 500 ng of total RNA for each PCR reaction. Amplification of both the viral target and endogenous cellular control target was performed as described previously [1-4], except all PCRs were performed in a 7900HT SDS thermal cycler and 96-well optical reaction plates (Applied Biosystems). Amplification of viral E1\hat{ }E4 and TATA binding protein (TBP, endogenous cellular control) mRNA species was performed using a duplex format in 0.2 ml, 96-well PCR plates with a total reaction volume of 25 μl. Based on a total reaction volume of 25 μl for HPV E1\hat{ }E4, mix: 12.5 μl 2× master mix, 7.25 μl RNase free water, 1.0 μl each of the 3′ and 5′ E1\hat{ }E4 primers (100 μM) (table 2), 0.5 μl E1\hat{ }E4 probe (10 μM) and 0.25 μl RT mix. A total master mix can be made by multiplying the number of wells being used, including 2 wells for a no template control (Load 2.5 μl RNA in each well for E1\hat{ }E4.)

Based on a total reaction volume of 25 μl for the TBP internal control wells, mix: 12.5 μl 2× master mix, 9.0 μl RNase free water, 0.125 μl each of the 3′ and 5′ TBP primers (10 μM), and 0.25 μl RT mix. A total master mix can be made by multiplying the number of wells being used, including 2 wells for a no template control (Load 2.5 μl RNA per well for TBP). Once the plate has been set up, it is loaded into a BIO-RAD iQ5 Multi-color Real-Time qPCR machine. The following cycling conditions are utilized: 50° C. for 30 minutes, 95° C. for 15 minutes, followed by 42 cycles of 94° C. for 15 seconds and 54.5° C. for 1 minute.

Relative expression is calculated using the 2-Ct method, with the efficiency of both the housekeeping gene and the target gene being similar.

Complete descriptions of the method used in calculating the disinfection efficacy in these experiments are included in the following references, each incorporated herein by reference in its entirety:
1. Conway M J, Alam S, Christensen N D, Meyers C. Overlapping and independent structural roles for human papillomavirus type 16 L2 conserved cysteines. Virology. 2009; 393(2):295-303. Epub 2009/09/08. doi: S0042-6822(09)00490-5 [pii] 10.1016/j.virol.2009.08.010. PubMed PMID: 19733888; PubMed Central PMCID: PMC2763 999.
2. Conway M J, Alam S, Ryndock E J, Cruz L, Christensen N D, Roden R B, et al. Tissue-spanning redox gradient-dependent assembly of native human papillomavirus type 16 virions. J Virol. 2009; 83(20): 10515-26. Epub 2009/08/07. doi: JVI.00731-09 [pii] 10.1128/JVI.00731-09. PubMed PMID: 19656879; PubMed Central PMCID: PMC2753102.
3. Conway M J, Cruz L, Alam S, Christensen N D, Meyers C. Cross-neutralization potential of native human papillomavirus N-terminal L2 epitopes. PLoS One. 2011; 6(2):e16405. Epub 2011/02/25. doi: 10.1371/journal.pone.0016405. PubMed PMID: 21346798; PubMed Central PMCID: PMC3035607.
4. Biryukov J, Cruz L, Ryndock E J, Meyers C. Native human papillomavirus production, quantification, and infectivity analysis. Methods Mol Biol. 2015; 1249:317-31. doi: 10.1007/978-1-4939-2013-6 24. PubMed PMID: 25348317.

### Example 1

### Representative Method for the Inactivation of Infectious Proteins

In this example, a representative method for the inactivation of infection proteins (prions) is described.

Full details of the measurement of the efficacy of BriotechHOCl™ versus infectious proteins are provided in Hughson, A. G., Race, B., Kraus, A., Sangare, L. R., Robins, L., Contreras, L., Groverman, B. R., Terry, D., Williams, J., and Caughey, B. (2016). Inactivation of Prions and Amyloid Seeds with Hypochlorous Acid. PLoS Pathogens, 12(9), e1005914. http://doi.org/10.1371/journal.ppat.1005914, expressly incorporated herein by reference in its entirety.

Briefly, Real Time Quaking Induced Conversion (RT-QuIC) assays were used to demonstrate that immersion in BriotechHOCl™ eliminated all detectable prion-seeding activity for human Creutzfeldt-Jakob Disease (CJD) prions, bovine spongiform encephalopathy (BSE) prions, cervine chronic wasting disease (CWD) prions, and sheep scrapie and hamster scrapie prions, causing reductions of >10³ to 10⁶ fold in 5 minutes to 60 minutes of exposure. Transgenic mouse bioassays showed that all detectable hamster-adapted sheep scrapie infectivity in brain homogenates or on steel wires was eliminated. These results represent reductions of infectivity of approximately 10⁶ fold and 10⁴ fold, respectively. Inactivation of RT-QuIC activity correlated with free chlorine concentration in the HOCl solutions, and higher order aggregation and/or destruction of proteins generally, including prion proteins. Those preparations of unbuffered BriotechHOCl™ that contained approximately 2% NaCl showed superior efficacy over solutions that were isotonic with mammalian cells (i.e., approximately 0.85% NaCl). These solutions of unbuffered HOCl uncontaminated by the presence of other aqueous halogen species had similar effects on self-replicating amyloid proteins composed of human alpha synuclein and a fragment of human tau protein.

In a further experiment the contact time of prion-enriched brain preparations with BriotechHOCl™ was reduced to 5 min after heat stress treatment of the solution. A >5 log reduction in prion seeding activity was seen with the HOCl preparation incubated at 22° C., but a 4.0 log reduction for preparation heat treated at 70° C. These results indicated that although each of the preparations eliminated all detectable prion seeding activity with 1 h treatments, a slight loss of anti-prion activity was observed after HOCl was heat stressed by prolonged storage at 70° C. when assayed with 5-min treatments.

TABLE 1
Log reduction value (LRV) of prion activity
following 5-minute HOCl sample treatments.

| | Prion seeding activity | |
|---|---|---|
| Sample | log₁₀ SD50/mg brain | LRV |
| Mock treatment | 8.2 | |
| 127 ppm HOCl 70° C. (14 days) | ≤4.2 | ≥4.0 |
| 280 ppm, (Dec. 4, 2017) | ≤3.2 | ≥5.0 |
| RT, 280 ppm (Nov. 20, 2017) | ≤4.9 | ≥3.3 |

In a further experiment the exposure of scrapie prion proteins in hamster brain preparations was conducted at 50° C. for 5 minutes of contact. The level of inactivation of self-replicating prions was enhanced by approximately one order of magnitude with exposure at 50° C. compared to that achieved at room temperature.

### Example 2

### Representative Method for the Inactivation of Infectious Viruses: HPV

In this example, a representative method for the inactivation of an infection virus (HPV) is described.

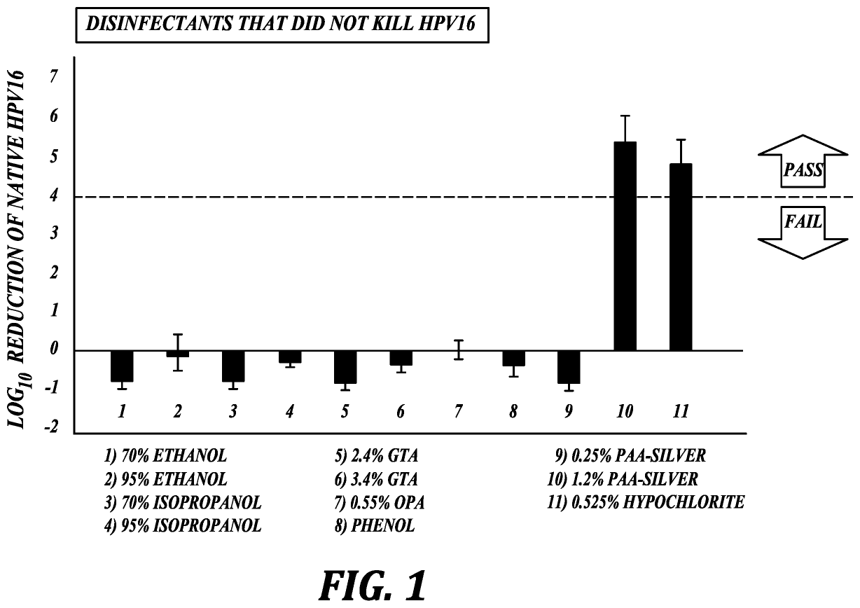

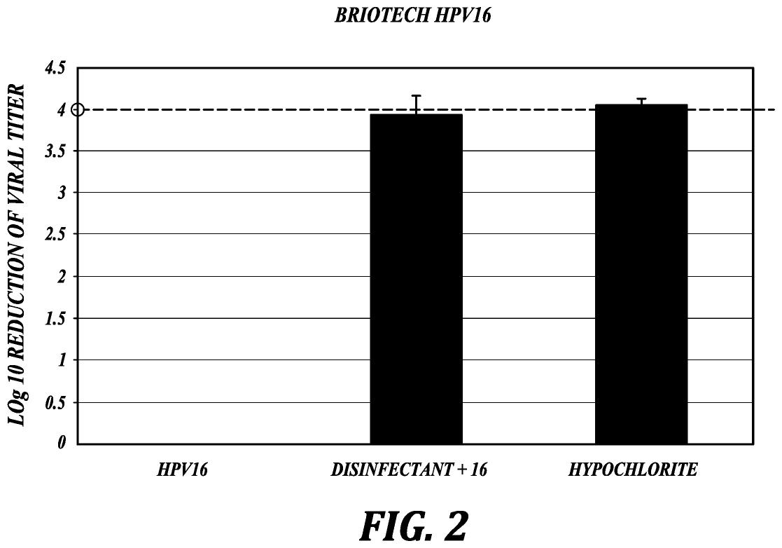

In this study of HOCl vs. HPV16, BriotechHOCL™ was just as effective as concentrated (20%) hypochlorite bleach. For comparison, FIG. 1 is a graph illustrating data on HPV disinfection with conventional agents, and FIG. 2 is a graph illustrating data on HPV disinfection with BriotechHOCl™. In experiments on the time course of exposure 99.9% reduction of HPV was shown at 5 minutes. At 30 minutes the reduction of 99.999% meets the gold standard for U.S. FDA approval of virucidal sterilants.

### Example 3

### Representative Method for the Inactivation of Infectious Viruses: MS2

In this example, a representative method for the inactivation of an infection virus (MS2) is described.

HOCl inactivated preparations of a highly resistant non-enveloped virus (MS2) to high levels in short contact times. The test was conducted using bacteriophages fixed to a 22 mm×22 mm×1 mm glass coupons with an exposure of 30 seconds, and 2 minutes, 5 minutes, and 10 minutes contact before neutralization with 0.05N sodium thiosulfate. The assay used was a modification of ASTM E2315. After exposure, the antimicrobial activity of the HOCl was terminated by immersion of the coupon into excess of neutralizer (20 mL of 0.05N sodium thiosulfate). The extent of the efficacy was measured by the formation of plaques on a lawn of Escherichia coli B(ATCC 15597-host bacteria) seeded using the top and bottom agar method with serial dilutions. Virus stock was harvested from cultures of host bacteria and quantitated by plate dilution (MS2 Stock Isolate≈1×10⁹-1×10¹⁰ pfu/mL). The stock was diluted 1:1,000 and 80 μl of the suspension were placed unto the glass coupons and allowed to air dry for 60 minutes prior to challenge.

Results are shown in Table 2

TABLE 2
Anti-MS2 (Bacterophage) Properties of BriotechHOCl ™.

| | Contact | MS2 | MS2 | | Percent |
|---|---|---|---|---|---|
| ID | Time | Challenge | Recovered | LRV | Kill |
| MS2 | 0 minutes | ND | 34,800 pfu | 0 | 0 |
| HOCl | 2 minutes | 34,800 pfu | 0 pfu | 4.54 | 100 |
| HOCl | 5 minutes | 34,800 pfu | 0 pfu | 4.54 | 100 |

### Example 4

### Representative Method for the Inactivation of Infectious Agents

In this example, a representative method for the inactivation of infection agents is described.

HOCl and HOBr inactivated Candida albicans yeasts in vitro to high levels in short contact times.

Tests were conducted using a standard ASTM E2315 method suspension protocol. Candida albicans was cultured from an inoculum from ATCC by an independent microbiology testing laboratory. Results are shown in Table 3 below. The contact time was 15 seconds.

TABLE 3
Anti-Candida yeast Properties of BriotechHOCl ™.

| | Log | | | |
|---|---|---|---|---|
| | Reduc- | Elimi- | | |
| | tion | nation | Testing | Testing |
| Pathogen | Value | % | Date | Site |
| Acinetobacter | 5.0 | >99.999% | 2 Jun. | Northwest Regional |
| baumannii | | | 2016 | Center of Excellence |
| | | | | & Emerging Infectious |
| | | | | Diseases Research, |
| | | | | Univ. of Washington |
| Aspergillus | 6.41 | >99.999% | 3 Aug. | Pacific Northwest |
| niger | | | 2016 | Microbiology Services |
| Bacillus | | >99.999% | 3 Aug. | Pacific Northwest |
| subtilis | | | 2016 | Microbiology Services |
| Candida | | >99.999% | 20 Nov. | Pacific Northwest |
| albicans | | | 2015 | Microbiology Services |

### Example 5

### Representative Method for the Treatment of Yeast Infection

In this example, a representative method for the treatment of a vaginal yeast infection in a subject is described.

A 30 year-old woman with a history of persistent vaginal yeast infection over 7 months, self-administered several ounces of BriotechHOCl™ after unsatisfactory experiences with conventional OTC and prescription pharmaceutical medications.

Intravaginal instillation of BriotechHOCl™ resulted in subsidence of the discharge and complete relief of irritation and elimination of all the signs of infection within 72 hours.

### Example 6

### Representative Method for the Treatment of Mucosal Infection

In this example, a representative method for the treatment of a chronic nasal mucosal infection in a subject is described.

A 49 year-old woman had a decade long history of chronic nasal mucosal infection, and associated symptoms of mucosal epithelial discharge, congestion of the nasal passage surfaces and nasopharyngeal discomfort. Tolerable control could only be achieved with regular antibiotic treatment regimens and antihistamines applied topically. Self-administration of two ounces of BriotechHOCl™ by instillation of the solution into the nasal passages resulted in complete cessation of all symptoms, with discomfort relief within hours, and no recurrence.

### Example 7

### Representative Method for the Treatment of Herpes Labialis

In this example, a representative method for the treatment of Herpes Labialis in a subject is described.

A 22 year old male had a history of Herpes Labialis, with recurrences 3-5 times/year, usually peri-labially, occasionally affecting nasal mucosa. Lesions appearing during episodes resolved slowly, typically over 2+ weeks, after an initial local tingling irritation phase. Self-administration of BriotechHOCl™ topically via spray 4-5 times a day led to rapid reduction in pain and redness. Formed sores, once open, respond in 4-5 days, with complete resolution in a week to 10 days. Habitual HOCl use has reduced the frequency of recurrences, now in the 1-2 per year range.

### Example 8

### Cytotoxicity of a Representative Hypohalous Acid Preparation: Briotechhocl™

Cytotoxicity of BriotechHOCl™ was tested according to established ASTM methods 10993-5, and 10993-12 using mouse cells by Product Safety Laboratories in Dayton, N.J., test #47677. The results showed that under the conditions of the test the HOCl solution showed no toxicity to NTCT L 929 mammalian cells. GLP tests performed on intact skin of guinea pigs and rabbits were performed by Pacific Biolabs, Hercules, Calif. The results showed no direct toxic effects on animal skin and caused no sensitization upon subsequent exposure to BriotechHOCl™.

While illustrative embodiments have been illustrated and described, it will be appreciated that various changes can be made therein without departing from the spirit and scope of the disclosure.

1. A method for inactivating an infectious agent that is a resistant virus, a cancer-causing virus, a chemically-resistant non-enveloped virus, or an infectious agent present in a mucous membrane or epithelial surface, comprising contacting an infectious agent with a bufferless, electrolyzed, hypohalous acid composition.

2. The method of claim 1, wherein the infectious agent is an infectious microbe.

3. The method of claim 2, wherein the infectious microbe is a virus, a bacterium, a fungus, or a protozoa.

4. The method of claim 1, wherein the infectious agent is an infectious protein.

5. The method of claim 4, wherein the infectious protein is a self-replicating protein.

6. The method of claim 4, wherein the infectious protein is a prion.

7. The method of claim 6, wherein the prion is an agent of Creutzfeldt Jakob Disease, Bovine Spongiform Encephalopathy, Chronic Wasting Disease, Scrapie, Alzheimer's Disease, Parkinson's Disease, and Amyotrophic Lateral Sclerosis.

8. The method of claim 1, wherein the infectious agent is a microbial pathogen.

9. The method of claim 8, wherein the microbial pathogen is a Gram negative bacterium.

10. The method of claim 8, wherein the microbial pathogen is Acinetobacter baumannii, Escherichia coli, Escherichia coli 0157 Pseudomonas aeruginosa, Salmonella choleraesuis, Shigella flexneri, Escherichia coli NDM-1, Klebsiella pneumonia, Yersinia enterocolitica, Proteus vulgaris, Neisseria, Chlamydia, or Listeria.

11. The method of claim 8, wherein the microbial pathogen is a Gram positive bacterium.

12. The method of claim 8, wherein the microbial pathogen is Bacillus subtilis, Staph epidermidis, MRSA (Staph. aureus), Enterobacter cloacae, or Enterococcus VRE.

13. The method of claim 8, wherein the microbial pathogen is a fungus.

14. The method of claim 8, wherein the microbial pathogen is Candida albicans or Aspergillus niger.

15. The method of claim 8, wherein the microbial pathogen is a virus.

16. The method of claim 8, wherein the microbial pathogen is Coronavirus [Human, OC43], Human Papilloma Virus (HPV), or MS-2.

17. The method of claim 1, wherein the composition has been thermally stressed prior to use.

18. The method of claim 1, wherein the composition has been stored for a prolonged period of time prior to use.

19. The method of claim 1, wherein the composition further comprises stabilizers.

20. The method of claim 1, wherein the composition is a solution, or a spray, a fog, a mist, or an aerosol of droplets (e.g., micronized droplets in the submicron size range and aerosolized droplets), or a gel, or a viscous liquid.

21. The method of claim 1, wherein contacting with the composition comprises contacting from one second to several hours.

22. The method of claim 1, wherein contacting with the composition comprises contacting at room temperature.

23. The method of claim 1, wherein contacting with the composition comprises contacting at a temperature in the range from about room temperature to about 80° C.

24. The method of claim 1, wherein the hypohalous acid composition is a hypochlorous acid composition.

25. The method of claim 1, wherein the hypohalous acid composition is an aqueous hypochlorous acid composition having a hypochlorous acid concentration from about 5 to about 500 mg/L, a pH from about 3.2 to about 6.0, an oxidative reduction potential (ORP) of about +1000 millivolts, and containing from about 0.85% to about 2.0% by weight chloride salt based on the total weight of the composition.

26. The method of claim 1, wherein the hypohalous acid composition is an aqueous hypochlorous acid composition having a hypochlorous acid concentration from about 80 to about 300 mg/L, a pH from about 3.8 to about 5.0, an oxidative reduction potential (ORP) of about +1100 millivolts, and containing from about 0.85% to about 2.0% by weight chloride salt based on the total weight of the composition.

27. The method of claim 1, wherein the hypohalous acid composition is an aqueous hypochlorous acid composition having a hypochlorous acid concentration from about 80 to about 300 mg/L, a pH from about 4.0 to about 4.3, an oxidative reduction potential (ORP) of about +1138 millivolts, and containing from about 0.85% to about 2.0% by weight chloride salt based on the total weight of the composition.

28. The method of claim 1, wherein the hypohalous acid composition is a hypobromous acid composition.

29. The method of claim 1, wherein the hypohalous acid composition is an aqueous hypobromous acid composition having a hypobromous acid concentration from about 10 to about 300 mg/L, a pH from about 3 to about 8.5, an oxidative reduction potential (ORP) of about +1000 millivolts, and containing from about 0.85% to about 2.0% by weight chloride salt based on the total weight of the composition.

30. The method of claim 1, wherein the hypohalous acid composition is an aqueous hypobromous acid composition having a hypobromous acid concentration from about 5 to about 350 mg/L, a pH of about 7 to about 8, an oxidative reduction potential (ORP) of about +900 millivolts, and containing from about 0.85% to about 2.0% by weight chloride salt based on the total weight of the composition.

31. The method of claim 25, wherein the chloride salt is an aqueous soluble chloride salt selected from sodium chloride, potassium chloride, magnesium chloride, and ammonium chloride.

32. The method of claim 25, wherein the chloride salt is sodium chloride.

33. The method of claim 25, wherein the composition contains about 2.0% by weight chloride salt based on the total weight of the composition.

34. The method of claim 25, wherein the composition contains about 2.0% by weight sodium chloride based on the total weight of the composition.

35. The method of claim 25, wherein the composition does not contain a detectable amount of aqueous oxidative chlorine other than a hypohalous acid.
